(19) United States
(12) Patent Application Publication
(10) Pub. No.: US 2026/0263086 A1
(43) Pub. Date: Sep. 10, 2026

(54) IMPLANT SITE PREPARATION SYSTEM AND METHOD THEREOF

(71) Applicant: MERIL HEALTHCARE PVT. LTD, Vapi (IN)

(72) Inventors: VIJAY C. BOSE, Chennai (IN); DARSHAN A. LAD, Bilimora (IN); JIMESH B. GANDHI, Bilimora (IN); PRATIK M. PANCHAL, Vapi (IN); LATHEESH KV, Kannur (IN)

(73) Assignee: MERIL HEALTHCARE PVT LTD, Vapi, GJ (IN)

(21) Appl. No.: 19/491,339

(22) PCT Filed: Jun. 7, 2024
(86) PCT No.: PCT/IN2024/050707

§ 371 Date: Dec. 8, 2025

(30) Foreign Application Priority Data
Jun. 7, 2023 (IN) 202321039117

(51) Int. Cl. A61B 17/16; A61B 17/17; A61B 17/92

(52) U.S. Cl. CPC A61B 17/164; A61B 17/175; A61B 17/92

(57) ABSTRACT

An implant system and a method for accurate placement of an implant is disclosed. The method includes the step of performing a primary provisional reaming (PP reaming) in a bone to obtain a cavity having a depth corresponding to which a maximum range of motion of a joint is achieved, indicating a soft tissue balance. The method further includes establishing a bone reference mark (BRM) corresponding to a length of a stem of a trial for which maximum range of motion of the joint is achieved, the length of the stem corresponds to the depth of the cavity; and performing a secondary scratch fit reaming (SS reaming) in the bone cavity w.r.t to the diameter of the implant till a scratch fit of an implant is achieved.

### FILED OF THE INVENTION

The present disclosure relates to an implant system for inserting an implant within a bone. More specifically, the present disclosure discloses the implant system and method thereof for accurate placement of the implant.

### BACKGROUND OF THE INVENTION

Naturally, a hip joint includes a femur bone (thigh bone) coupled to a pelvic bone via a ball and socket joint. People suffering from deteriorating hip joints undergo a hip replacement surgery. In the hip replacement surgery, the hip joint is completely or partially replaced by a synthetic implant.

A conventional implant or stem prosthesis, used to reinforce the femur bone in the hip replacement surgery, includes an axially extending stem portion and a neck portion that is at least partially disposed at an angle with respect to the stem portion. The stem prosthesis is inserted within a bone cavity of the femur bone. The neck portion indirectly connects the stem portion to the pelvic bone.

Conventionally available stem prostheses are one of a monoblock type or a modular type. As the name suggests, the monoblock type stem prosthesis is an integral structure of the neck portion and the stem portion. Monoblock stem prosthesis are easy to insert and have no risk of breakage. However, the monoblock stem prosthesis suffer from higher incidents of subsidence and inability to restore soft tissue tension.

In contrast, the modular type stem prosthesis has a removably coupled neck portion to the stem portion. The modular stem prosthesis is relatively more resistant to subsidence and provides better ability to achieve soft tissue balance. However, they are more cumbersome to insert within the bone and are prone to break where the stem portion and the neck portion are coupled.

To implant any of the stem prosthesis, a hip replacement surgery is performed by a surgeon which is one of the most complicated procedures to perform. The procedure requires a correct size bone cavity to be reamed within the bone and a correct size of the implant (with respect to the size of the bone cavity) to be inserted to achieve soft tissue balance.

Surgeons commonly achieve implant fixation within the bone cavity with a one-step reaming/trialing process. Conventional revision stem designs use the head center as the reference level for the reamer, trial, and/or implant. As the femoral head is spatially away from the bone, head height level in relation to bone cannot be estimated accurately, thus resulting in mismatch between the offset and seating level of the trial and the implant. The said mismatch leads to inferior soft tissue balance. The aforesaid problems, often times, motivate the surgeons to use modular implants and in turn, sacrifice the advantages of the monoblock implants.

There thus exists a need to provide a method and an implant system for overcoming the aforesaid problems.

### SUMMARY OF THE INVENTION

Particular embodiments of the present disclosure are described herein below with reference to the accompanying drawings, however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present disclosure relates to an implant system and a corresponding method of accurately placing an implant inside a bone cavity (for example, inside a femur bone) using the said implant system. The components of the implant system include, for example, one or more trials, an implant extractor, an implant applicator, a canal finder, a reamer, etc. The method of the present disclosure includes two or more stages of reaming and trialing.

In an exemplary embodiment, the method includes the step of performing a primary provisional reaming (PP reaming) in a bone to obtain a cavity having a depth corresponding to which a maximum range of motion of a joint is achieved, indicating a soft tissue balance. The method further includes establishing a bone reference mark (BRM) corresponding to a length of a stem of a trial for which maximum range of motion of the joint is achieved, the length of the stem corresponds to the depth of the cavity; and performing a secondary scratch fit reaming (SS reaming) in the bone cavity w.r.t to the diameter of the implant till a scratch fit of an implant is achieved.

In another embodiment, an implant system for accurate placement of an implant is disclosed. The implant system includes one or more trials; a reamer comprising a plurality of aligners provided on its body, wherein the reamer is configured to perform a primary provisional reaming (PP reaming) in a bone to obtain a cavity having a depth corresponding to which a maximum range of motion of a joint indicating a soft tissue balance, wherein the reamer is configured to perform a secondary scratch fit reaming (SS reaming) in the bone cavity w.r.t to the diameter of the implant till a scratch fit of an implant is achieved; and wherein the aligners establish a bone reference mark (BRM) corresponding to a length of a stem of a trial of the one or more trials for which maximum range of motion of the joint is achieved, the length of the stem corresponds to the depth of the cavity.

### BRIEF DESCRIPTION OF DRAWINGS

For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 illustrates an exemplary flowchart of a method 1000 in accordance with an embodiment of the present disclosure.

FIG. 2 illustrates an implant extractor 210 coupled to an implant 200 in accordance with an embodiment of the present disclosure.

FIGS. 3, 3a and 3b illustrate a reamer 300 or portions thereof in accordance with an embodiment of the present disclosure.

FIG. 4 illustrates the reamer 300 within a bone cavity 402 in accordance with an embodiment of the present disclosure.

FIG. 5a-5c illustrates coupling of the neck 504 with the stem 502 of a trial 500 in accordance with an embodiment of the present disclosure.

FIG. 6a illustrates a cross-sectional view of the trial 500 with a neck 504 concentric with a stem 502 of the trial 500 in accordance with an embodiment of the present disclosure.

FIG. 6b illustrates a cross-sectional view of the trial 500 with the neck 504 eccentric with the stem 502 of the trial 500 in accordance with an embodiment of the present disclosure.

FIG. 7 illustrates the trial 500 coupled to an implant applicator 700 in accordance with an embodiment of the present disclosure.

FIGS. 8a and 8b illustrate a bone reference mark (BRM) in reference to the trial 500 accordance with an embodiment of the present disclosure.

FIGS. 9a and 9b illustrate alignment of the reamer 300 with the bone reference mark (BRM) in accordance with an embodiment of the present disclosure.

FIG. 10 illustrates a final trial 800 coupled to the implant applicator 700 in accordance with an embodiment of the present disclosure.

FIG. 11 illustrates placement and coupling of a definitive implant 1100 in accordance with an embodiment of the present disclosure.

FIG. 12 illustrates a head 1106 being coupled to the implant 1100 by using the impactor in accordance with an embodiment of the present disclosure.

### DETAILED DESCRIPTION OF DRAWINGS

Prior to describing the disclosure in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like. Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

The present disclosure relates to an implant system and method thereof for accurate placement of an implant. The first stage (or a primary provisional reaming stage) of the method helps to obtain a bone reference mark (BRM) and establish a soft tissue balance. The second stage (or secondary scratch fit reaming stage) of the method helps to establish adequate scratch fit of the implant within the bone cavity. using a reamer(s) and/or trial(s)

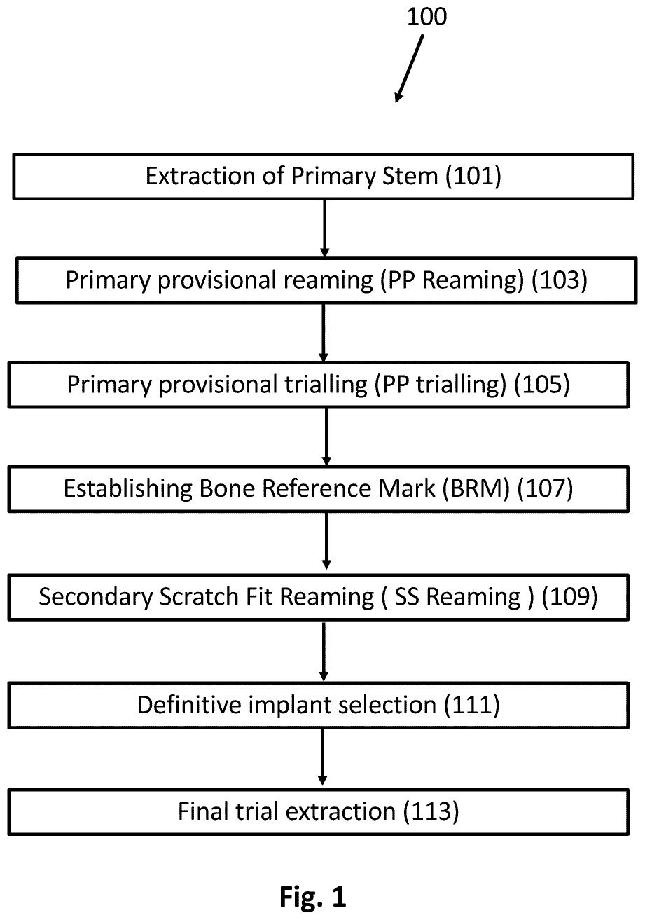

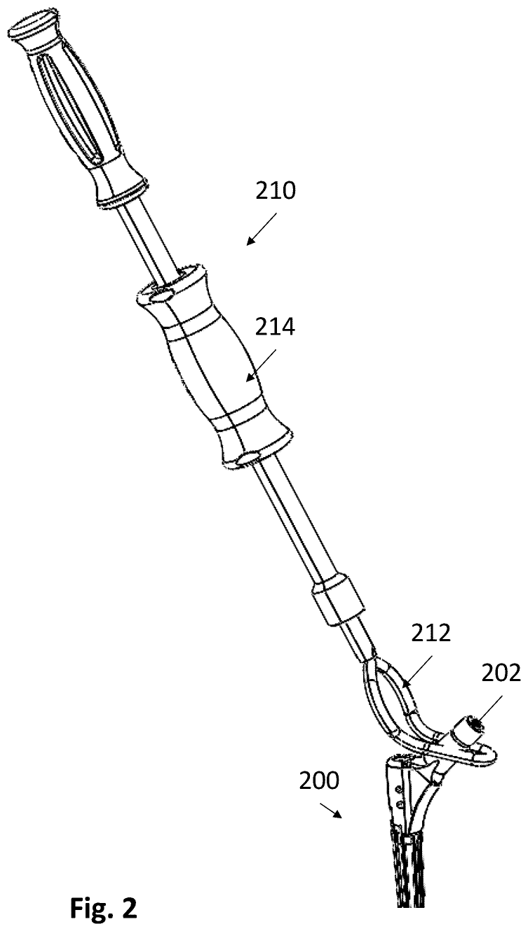

FIG. 1 details a method for preparing a bone cavity configured to receive an implant. The method commences with an optional step 101 directed to checking presence of an old implant 200 in the bone. For example, a medical expert may confirm if an old implant 200 is present inside the bone of a patient. If an old implant 200 is found, the surgeon may use an implant extractor to extract the old implant 200 from the bone cavity. An exemplary implant extractor 210 is depicted in FIG. 2.

The implant extractor 210 includes a grabbing element such as a connector 212. The connector 212 is provided at the distal end of the implant extractor 210. The connector 212 is configured to couple with the upper portion of the old implant 200 as depicted in FIG. 2. In an embodiment, the connector 212 is coupled to a neck 504 of the old implant 200. For example, the connector 212 is configured to lock onto the neck 504 of the old implant 200. Further, an axial force is delivered to the implant extractor 210 by using a suitable device such as a slap hammer 214. The slap hammer 214 provides blows to the implant extractor 210 leading to extraction of the old implant 200 from the bone cavity (not shown).

Optionally, further in an embodiment, a canal finder (not shown) with a sharp tip may be required to establish the correct direction of medullary canal. The canal finder is configured avoid any mismatch between a mechanical axis of the bone and a reamer axis of the bone. Alternatively, any other suitable instrument known in the art can be utilized at this stage of the method.

At step 103, a primary provisional reaming (PP reaming) of a bone cavity is performed. The primary provisional reaming is the first stage bone cavity reconfiguration of a bone 400. The primary provisional reaming reams the bone cavity up to a required depth for achieving a soft tissue balance. The soft tissue balance refers to ensuring an adequate range of motion of a trial/implant of the joint.

Further, a bone 400 is reamed in the ascertained direction defined by the canal finder to create a canal (or bone cavity) 402 using a reamer. Alternately, in case a bone cavity is present, the direction of reaming may not be needed to be ascertained.

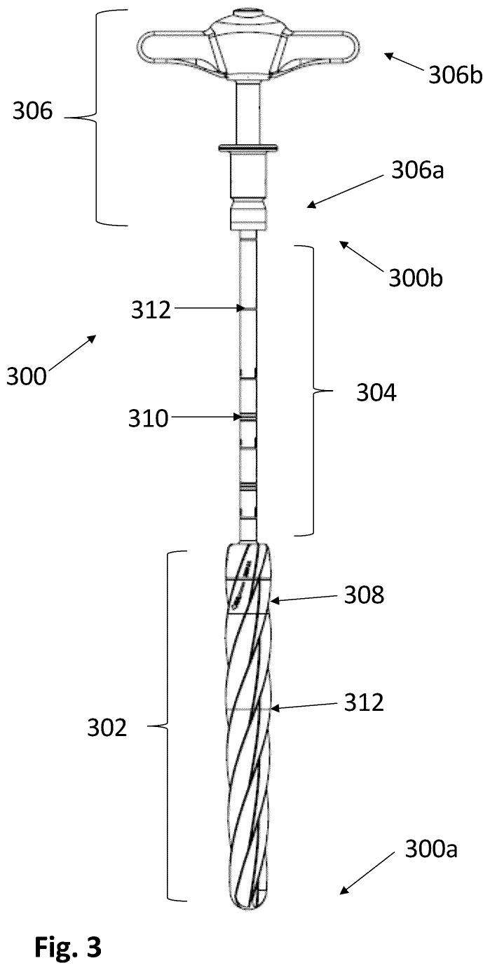

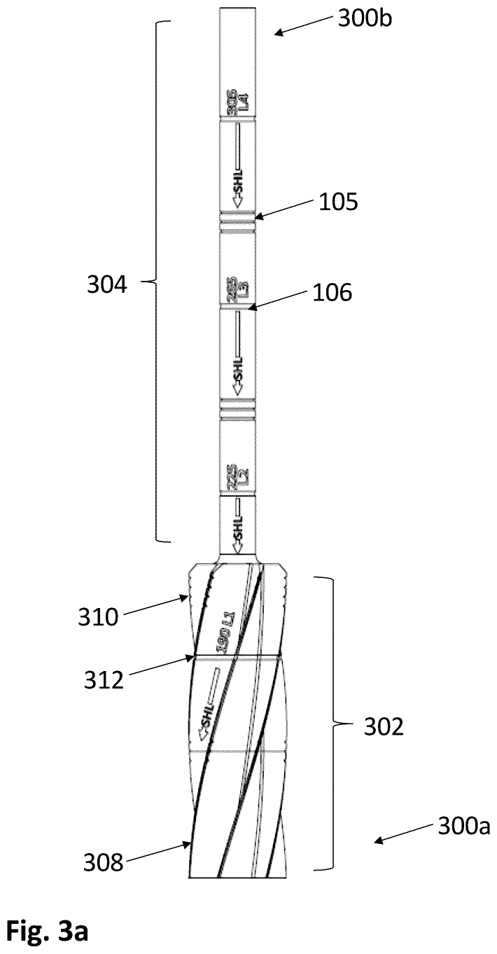

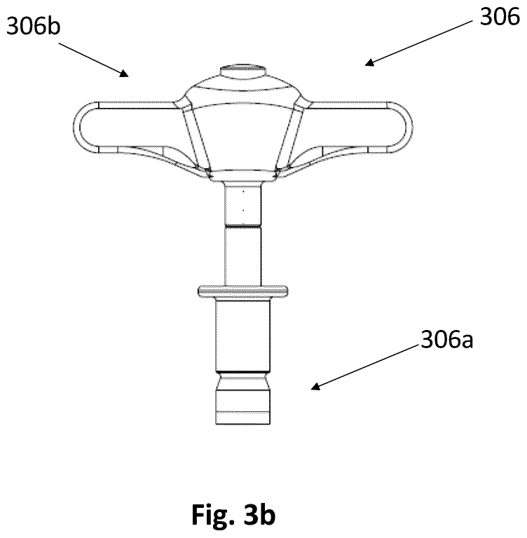

An exemplary reamer 300 is described in FIG. 3. The reamer 300 is configured to ream a bone cavity 402 in the medullary canal of the bone 400. Alternatively, any conventional reamer or any other suitable reaming instrument (powered or manual) can be utilized for reconfiguration of the bone cavity 402 at this stage of the method.

The reamer 300 includes a distal end 300a and a proximal end 300b (FIG. 3). In an embodiment, the reamer 300 includes a body 302, an elongated member 304 and a handle 103. The body 302 of the reamer 300 is disposed towards the distal end 300a of the reamer 300, while the handle 306is disposed towards the proximal end 300b with the elongated member 304 in between. The reamer 300 can be made from any suitable bio compatible material such as stainless steel, CoCr, titanium, or metal alloy etc. In an embodiment, the reamer 300 is made from stainless steel 17-4 PH.

The body 302 of the reamer 300 includes a cylindrical profile. Alternatively, the body 302 can be of other suitable shape. The body 302 of the reamer 300 includes a plurality of flutes 308 (FIG. 3a). The flutes 308 can extend around the surface in a pre-defined pattern or in a straight line. The flutes 308 extend at least partially along the length of the body 302 of the reamer 300. In an embodiment, the flutes 308 extend spirally along the entire length of the body 302 of the reamer 300. The flutes 308 reduce the force required during reaming of the bone 400 by the user.

The flutes 308 on the body 302 may be provided with a plurality of aligners 310. The aligners 310 indicate shoulder height level (SHL) of an implant (or trial) having stems with varying diameter. The elongated member 304 of the reamer 300 may also be provided with aligners 310. The aligners 310 provided on the elongated member 304 and the flutes 308 of the body 302 may be structurally same or different.

The aligners 310 (FIG. 3a) include at least one colored mark band. For example, an aligner 310 may include three colored mark bands, each band depicting a shoulder height of a different stem. This is so as different implants (or trials) have different stem sizes. In an embodiment, the aligners 310 include a different color configured for a specific range of stem sizes of implants (or trials). In an embodiment, the aligner 310 includes three markings a first marking, a second marking and a third marking. The first marking is configured to depict shoulder height of implants (or trials) for stem sizes ranging from 014 mm to 017 mm. The second marking is configured to depict shoulder height of implants (or trials) for stem sizes ranging from 018 mm to 021 mm. The third marking is configured to depict shoulder height of implants (or trials) for stem sizes ranging from 022 mm to 025 mm.

Further, the body 302 and the elongated member 304 of the reamer 300 is provided with a plurality of grooves 312. The grooves 312 indicate the length of the stems of the implant (or trials).

The handle 306 provided at the proximal end 300b of the reamer 300 may be coupled fixedly or detachably to the elongated member 304. The handle 306 includes a coupling end 103a and a holding end 103b. Towards the coupling end 103a, the handle 306 is coupled to the elongated member 304 of the reamer 300. In an embodiment, the handle 306 is detachably coupled to the reamer 300. The handle 306 and the reamer 300 can be coupled using any suitable mechanism such as snap fit, bayonet, hudson coupling, quick connect coupling mechanism, etc. The handle 306 includes a T-shape (FIG. 3b). Though alternate embodiments can include any other suitable shape.

In an embodiment, the handle 306 of the reamer 300 is rotated by applying a twisting force manually towards the holding end 103b, enabling the surgeon to ream the canal 402. At the start of the process, the initial diameter of the selected body of the reamer 300 (or reamer body 302) may be at least 12 mm. The process of reaming is done manually which provides a feel of the canal to the surgeon. The process of reaming is repeated using reamer body 302 of progressively increasing diameter. This process is repeated until the surgeon finds a ‘catch’ in the intramedullary canal with a reamer body 302, sufficient for stability of the trial during trial insertion.

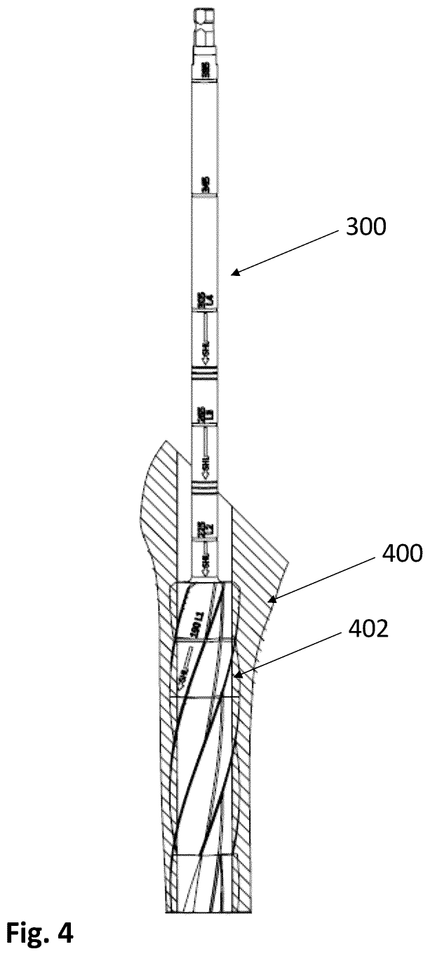

Additionally or optionally, before commencing the process of reaming, a length of the stem of the implant (or trial) is determined. The length may be determined using an x-ray image or an image from any other suitable imaging modality which can depict the whole region of interest in a single frame, of the femur bone 400. The size of an initial reamer body 302 required to ream the bone cavity 402 (FIG. 4) may be selected from variables (say length, and diameter) derived from the image.

Further, in an embodiment, a tip of the trochanter (of the femur bone) is used as an approximate starting point for matching the level of tip which is marked on the reamer 300. The greater trochanter is matched with the nearest stem length using the grooves 312 provided on the reamer 300. The matching is essential for ensuring alignment, stability and support of the implant in accordance with the patient's bone structure. The said alignment marks the starting point of the trial reduction process.

At step 105, a primary provisional trialling (PP trialling) in the bone cavity is performed. It is to be noted that the primary provisional trialling process may involve inserting one or more trials one after the other till a perfect fit trial for the bone cavity is found.

In this step, a trial implant (or trial) 200 corresponding to which the reamer body 302 is caught in the intramedullary canal (as described above), is selected. This is referred as perfect fit trial. The length of the stem of the perfect fit trial corresponds to the length of the reamer body 302 aligned with the tip of the trochanter, caught in the intramedullary canal. Or, the length of the perfect fit trial corresponds to the length of a groove 312 of the reamer body 302 caught in the intramedullary canal. The perfect fit trial in the bone cavity helps to achieve proper soft tissue balance. Further, the perfect fit trial 500 obtained during primary provisional reaming provides a scratch fit regarding the length of the stem of the implant to be implanted inside the bone cavity. That is, the primary provisional reaming establishes the depth of the bone cavity of the bone that ensures soft tissue balance. The primary provisional reaming does not provide scratch fit in terms of the width of the prosthesis needed. The reason being to enable further adjustments as required when the implant is inserted which in turn may be marginally larger (say, 1-2 mm) than the stem of the trial 500. It is to be noted that steps 103 and 105 may be repeated one or more times to obtain proper soft tissue balance regarding the length of the definitive implant to be implanted.

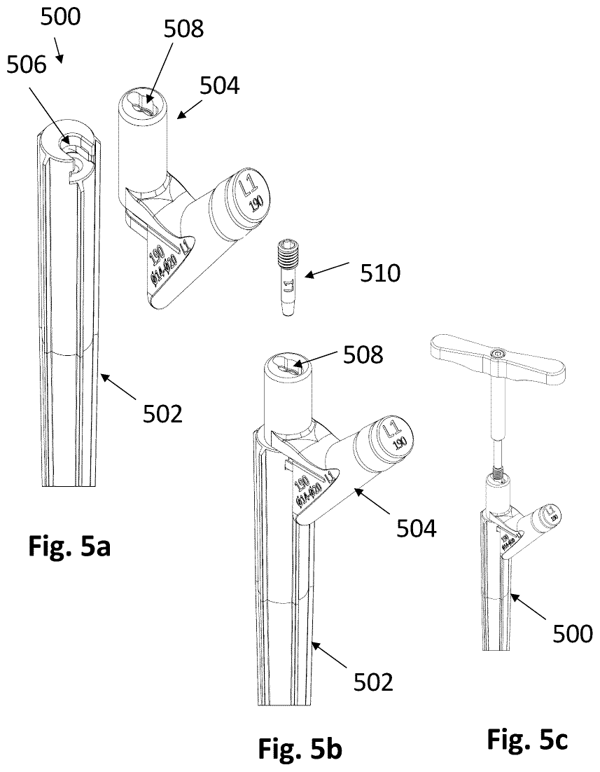

In an exemplary embodiment of a trial 500 as depicted in FIG. 5a-5c, the trial 500 is assembled by coupling its components. Alternately, any conventional trial can be used for trial reduction, by an expert. The trial may be one of a monoblock trial or a modular trial. In the former case, the neck 504 and the stem 502 can be an integral structure, making the trial 500 monoblock. In the latter case, the neck 504 is removably coupled to the stem 502, making the trial 500 modular.

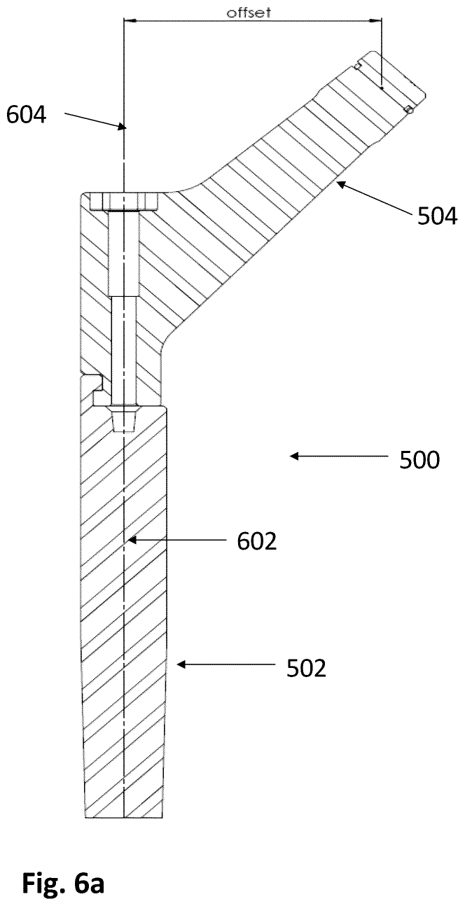

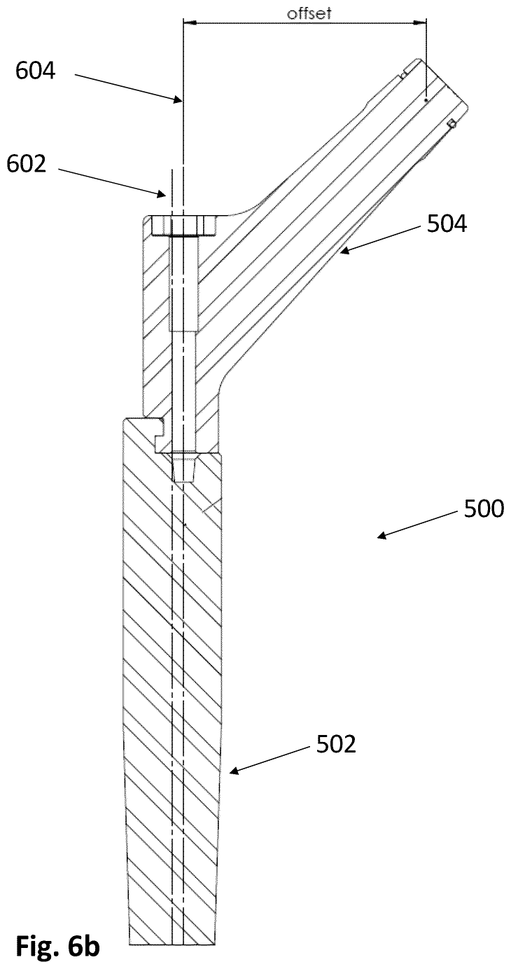

The trial 500 includes a stem 502 and a neck 504. The stem 502 includes a stem axis 602 and the neck includes an aperture axis 604. In an exemplary embodiment, the neck 504 is disposed concentric with the stem 502. In other words, the stem axis 602 of the stem 502 and the aperture axis 604 of the neck 504 have same center point or axis of rotation as seen in FIG. 6a. The stem 502 may have a pre-defined diameter. The neck 504 may have a pre-defined length. The length of the neck 504 selectively corresponds to the diameter of the stem 502. In other words, for a stem 502 having a pre-defined diameter, the neck 504 can have a selected range of length.

In an exemplary embodiment, as the diameter of the stem 502 increases and the length of the neck 504 increases, the neck 504 is disposed eccentric to the stem 502 (FIG. 6b). In other words, the neck 504 is laterally offset from the stem 502. Or, the stem axis 602 and the aperture axis 604 do not share the same center point or axis of rotation and are offset from each other as seen in FIG. 6b. The offset helps to prevent mismatch between the trial 500 and the implant, thus preventing the need to use a modular implant for last minute adjustments. In other words, the eccentric disposition of the neck 504 with respect to the stem 502 helps to achieve an exact offset of the (definitive) implant.

In case a modular trial is used in the process, the selected trial 500 is assembled. The stem 502 and the neck 504 can be coupled using any suitable mechanism such as fastening, T-slot locking, etc. In an embodiment, the stem 502 and the neck 504 of the trial 500 are provided with a T-slot locking mechanism corresponding to each other. In an exemplary embodiment, once the neck 504 having a desired length is coupled to the stem 502 having a desired diameter, a fastener 510 is used to secure the neck 504 with the stem 502.

The upper portion of the stem 502 of the trial may include a slot 506. The slot 506 is configured to receive the lower portion of the neck 504. The top portion of the neck 504 may include an aperture 508. The aperture 508 is configured to receive a fastener 510. Further, the fastener 510 is fastened using a fastening apparatus.

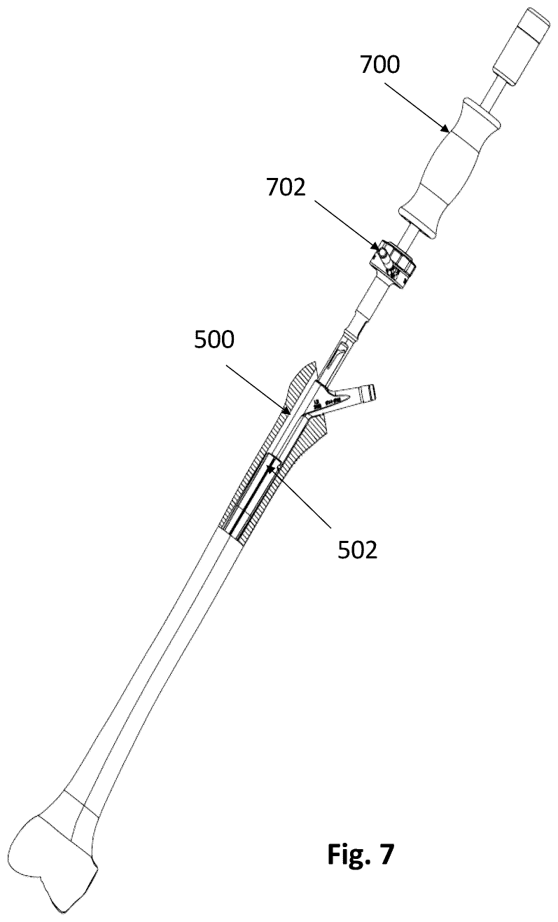

For insertion in the bone cavity, first anteversion of the trial 500 is determined. For this, the trial 500 is coupled with an exemplary implant applicator 700 (FIG. 7). It is to be noted that the implant applicator 700 as described is exemplary and any implant applicator can be utilized as per the teachings of the present disclosure. Alternately, an exemplary implant applicator 700 as described in Indian patent application number 202321039103, can be used.

Firstly, the trial 500 is coupled to a T-adaptor 702 using any suitable provided coupling mechanism. The T-adaptor 702 is configured with a variable anteversion guide. The T-adaptor 702 includes a plurality of marking slots. For example, the T-adaptor (not shown) includes 0°, 10°, 20°and 30°markings. The markings are provided with slots which are configured to couple with a guide member such as an anteversion alignment pin using any suitable coupling mechanism such as fastening, threaded mechanism, snap-fit etc. The guide member (anteversion alignment pin) can be coupled to the relevant angular holes (marking apertures) for the desired anteversion. Aligning the guide member to the longitudinal axis of the tibia, while the tibia bone is flexed and held perpendicular to the femur bone, automatically provides the particular anteversion angle for the trial 500. The trial 500 is coupled with the T-adaptor with variable anteversion guide and is impacted using a light mallet.

Once the anteversion of the trial 500 is determined, the trial 500 is inserted in the reamed canal 402 using the implant applicator. In an embodiment, an assembly of the trial 500 coupled with the implant applicator (or a part thereof), is placed in the canal 402 such that the free end of the trial 500 enters within the canal 402. In an embodiment, a light impactor is used to impact the implant applicator 700 (or a part thereof) to facilitate trial insertion within the bone cavity 402 of the bone 400. In an exemplary embodiment, the implant applicator 700 is used without the impactor, elongate member and the stoppers at the time of PP reaming.

Once the stem 502 of the trial 500 is inserted, it is important to maintain the soft tissue balance. Optimum soft tissue balance is determined by the trial reduction process. For example, if the soft tissues become tight after reduction, further reaming is performed by the surgeon to ensure that the trial stem can be sunk further in the canal 402 or if further reaming is not possible (due to medical conditions like sclerotic bone), a one size smaller trial may be used. On the contrary, if the soft tissues are very lax then, a reamer with the next bigger dimension of the reamer body 302 is used to further ream the canal 402. The trial having next bigger dimension is then inserted in the reamed canal 402. By adjusting in this manner, an appropriate height of the trial that achieves ideal soft tissue balance, is determined.

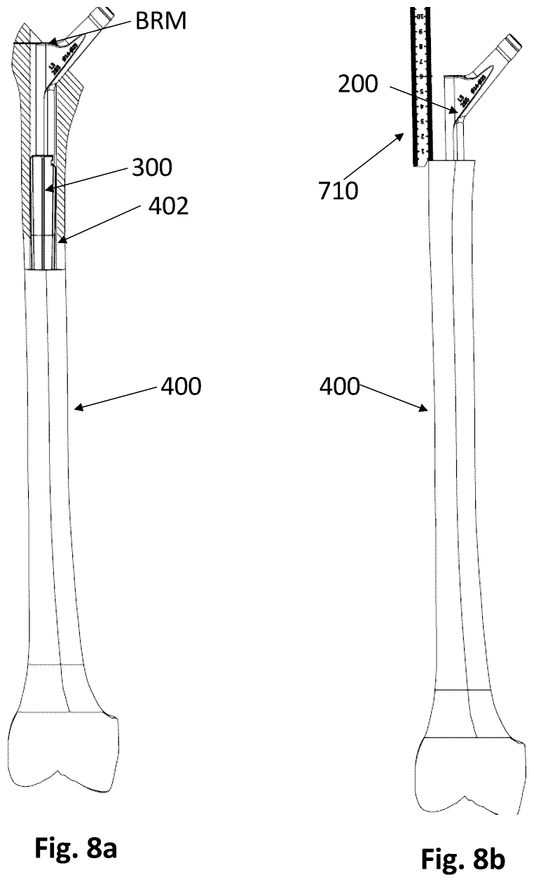

At step 107, a bone reference mark BRM w.r.t to the length is established. The height of the trial reflecting ideal soft tissue balance is selected and marked on the adjacent/proximal bone (as shown in FIG. 8a). This mark is referred to as a Bone Reference Mark (BRM). For example, the level of a shoulder ‘s’ of the trial in this position is marked on the adjacent bone using a suitable medical instrument such as a cautery tip. In case if the proximal bone is absent (FIG. 8b), the level of the shoulder ‘s’ may be measured from a fixed point on the distal bone using a specialized instrument such as, a ruler 710. An embodiment of a ruler 710 is depicted in FIG. 8b, however other marking system for marking BRM can be utilized.

The ruler 710 may include a cut-out of suitable shape such as a V or V-cut towards its initial end to facilitate the distal bone. The initial end of the ruler 710 with the V-cut is placed inside the distal bone and the shoulder level of the trial is marked on the scale using a suitable instrument such as a sterile marker pen indicating the bone reference mark (BRM).

At step 109, a secondary scratch fit reaming (SS reaming) on the bone cavity is performed. The SS reaming is commenced to achieve the soft tissue balance w.r.t to the circumference of the bone cavity. Earlier, during PP reaming the soft tissue balance w.r.t to the depth of the bone cavity or length of the stem of the implant and the BRM was achieved.

During SS reaming, the bone cavity 402 is further reamed using a reamer. The reamer may include a larger diameter than the one used in the PP reaming. The SS reaming is performed by the surgeon to fine tune the soft tissue balance by using a trial having a different neck, say of a larger diameter than the one used in PP reaming. The reamer with larger diameter reconfigures circumference of the bone cavity. The amount of reconfiguration of the circumference of the bone cavity may depend upon various factors such as bone's anatomy, offset to be achieved, a predefined relation between the dimensions of the implant, etc.

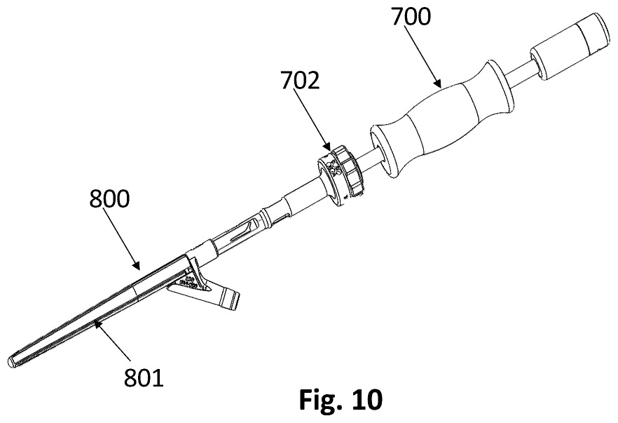

Further, during trailing (or trial reduction process) the surgeon may use a plurality of non-permanent versions of the implants to assess the various aspects required for an implant during implantation such as soft tissue balance, offset, biocompatibility of material, range of motion of the joint, etc. In an embodiment, the surgeon uses a trial 800 having similar specifications to a definitive implant. Alternatively, the trial 800 may include any other suitable configuration as per requirements. In other words, the final trial 800 (FIG. 10) selected after SS reaming is offset specific and matches the implant to be inserted within the femur bone for achieving soft tissue balance. This helps to eliminate the need to have last minute modularity (as in the case of modular implants) at the time of implanting the implant.

Soft tissue balance during trialing (or trial reduction) is translated to (definitive) implant as there is no variation in offset between the final trial 800 and the (definitive) implant. Further, trial reduction helps to achieve a tight scratch fit with a good canal fill for a length of trials for example, 5 to 7 cms. In other words, trial reduction (as described above) is again performed to obtain a soft tissue balance. This time, the soft tissue balance is achieved w.r.t to the bone cavity walls for attaining stability of the joint. In an embodiment, the tight scratch fit reaming is done with power instruments wherein the intramedullary canal is reshaped circumferentially to the same conical taper as that of the stem of an implant to be implanted (definitive implant) so that it achieves a cone in cone press fit.

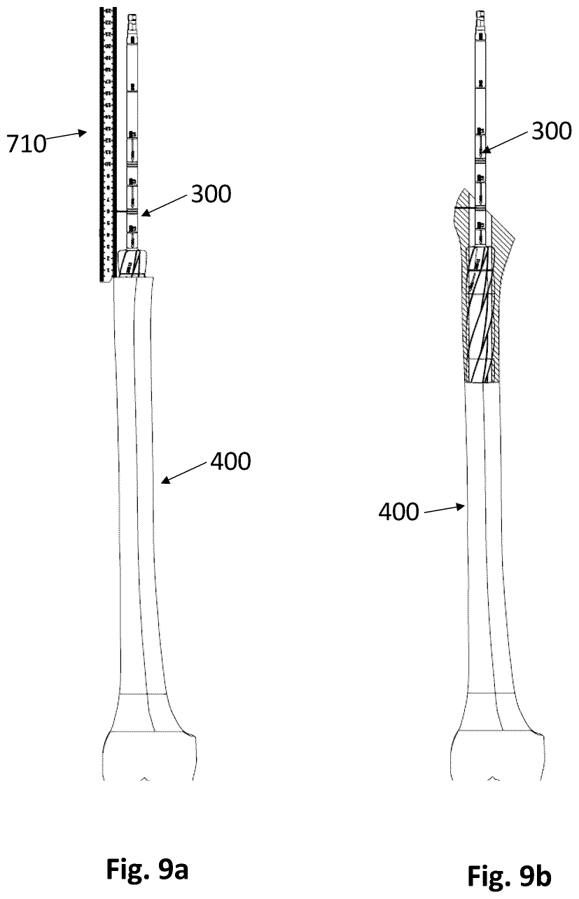

As shown in FIGS. 9a and 9b, further reaming is undertaken till the shoulder height level (SHL) (aligners 310) etched on the reamer body 302 is accurately matched with the BRM made post trial insertion. Alternatively or additionally, when proper scratch fit is achieved with respect to the reamed bone cavity based on the diameter selected in the PP reaming, the SS reaming can be concluded. In an exemplary embodiment, not shown, the canal fill and the length of fit of the trial is checked using a C-arm or any other suitable imaging mechanism.

At step 111, a definitive implant is selected. The specifications of the definitive implant correspond to the dimensions of a final trial via which a soft-tissue balance is achieved.

Further, the T-adaptor 702 is coupled to the trial 800 to extract the trial and thereafter, to seat the definitive implant in the bone cavity by hand, ensuring correct anteversion (FIG. 10). A lighter or heavier impact applicator 700 is coupled to the T-adaptor based on the bone type. Lighter stem applicator is selected if the bone is osteoporotic. Alternately, a heavier impact applicator 700 is selected, if the bone is sclerotic or normal. The impact applicator 700 is designed to deliver a fixed force which is independent of the surgeon. A stem 801 of the trial 800 is advanced with the impact applicator 700 till the stem 801 stops moving or resists the forward movement within the bone cavity. No additional force by using any external force delivering movement is required as, the scratch fit is done in such a way that the trial 800 is at or in proximity to the BRM. Further, excessive force on the impact applicator 700 can pose the risk of bone fracture.

Once the shoulder ‘s’ of a final trial 800 is in close proximity to the BRM obtained by PP reaming, final trial reduction takes place.

At step 113, the final trial 800 is extracted using the implant applicator 700 to make room for the implant. The implant is thereafter inserted.

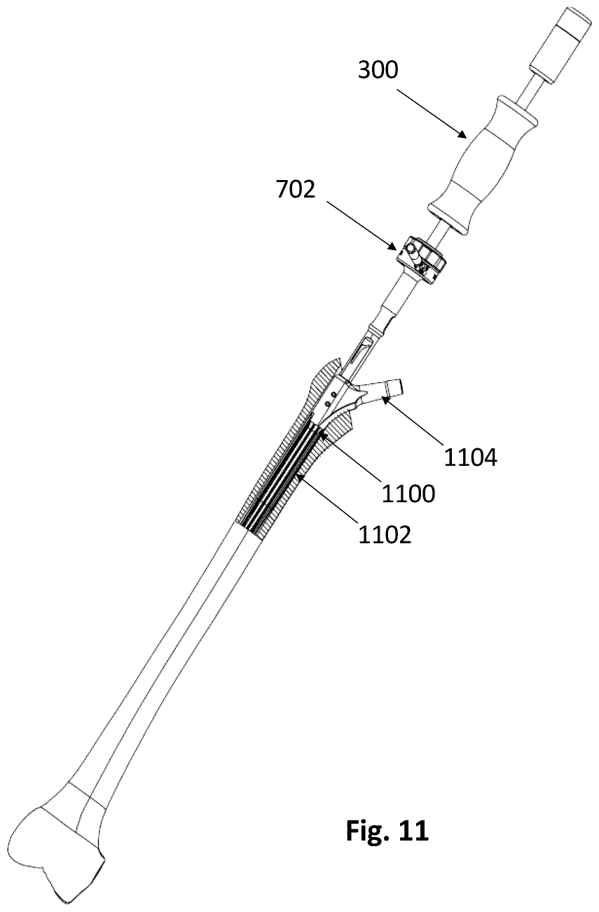

FIG. 11 depicts an exemplary implant 1100 coupled to the stem applicator 300. The implant 1100, selected basis the final trial 800, is inserted into the bone cavity 402 (as shown in FIG. 9a) using the implant applicator 700 as described in Indian patent application number 202321039103.

An exemplary implant is depicted in FIG. 11. The implant 1100 includes a stem portion 1102 and a neck portion 1104. In an exemplary embodiment, the stem portion 1102 and the neck portion 1104 of the implant 1100 form an integral structure. In an alternative embodiment, the stem portion 1102 and the neck portion 1104 of the implant 1100 are removably coupled to each other making the implant 1100 modular.

The implant 1100 is inserted inside the bone cavity using the stem applicator 300. Again, the T-adaptor 702 with variable anteversion slots guides the surgeon to achieve the desired and final anteversion of the stem 1102. The stem 1102 is seated such that any further advancement related to its position using the stem applicator 300 is not possible. The surgeon further achieves the confirmation of the scratch fit between the length and the circumference of the bone cavity and the implant 1100 as the neck portion 1104 is in proximity or at the BRM achieved in PP reaming.

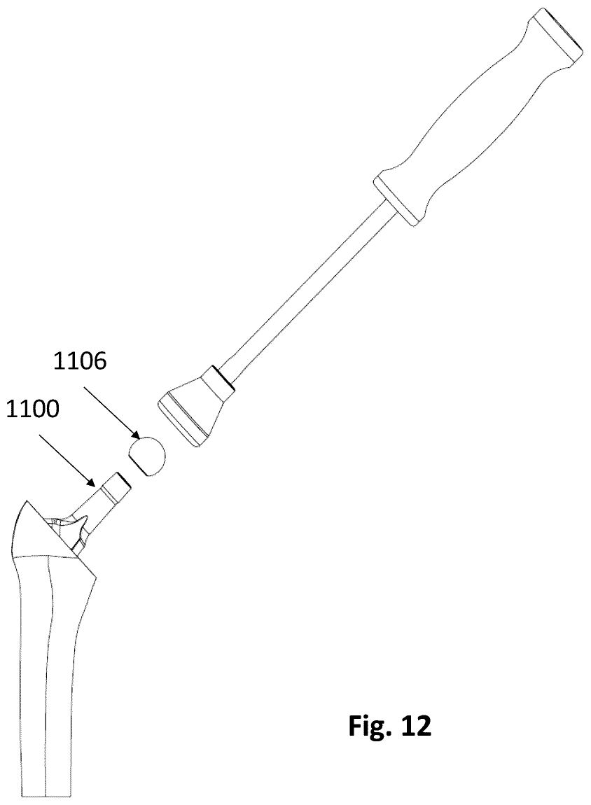

A head 1106 is impacted on to the neck of the implant 1100 (as shown in FIG. 12). In an exemplary embodiment, the head 1106 is mounted using slight rotation and locked in place by a light hammer blow on an impactor.

In alternate embodiments use of any other impactor or similar instrument required for impaction or fitting of the implant 1100 can be utilized at this stage of the method accordingly.

In an exemplary embodiment, not shown, the head 1106 is operationally coupled to a native socket of the pelvic bone or an implant disposed on the pelvic bone, thereby restoring the functionality of a natural hip joint.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A method for accurate placement of an implant, comprising:
performing a primary provisional reaming (PP reaming) in a bone to obtain a cavity having a depth corresponding to which a maximum range of motion of a joint is achieved, indicating a soft tissue balance;
establishing a bone reference mark (BRM) corresponding to a length of a stem of a trial for which maximum range of motion of the joint is achieved, the length of the stem corresponds to the depth of the cavity; and
performing a secondary scratch fit reaming (SS reaming) in the bone cavity w.r.t to the diameter of the implant till a scratch fit of an implant is achieved.

2. The method as claimed in claim 1 wherein, the step of performing the primary provisional reaming includes reaming the bone cavity using a reamer.

3. The method as claimed in claim 1 wherein, the step of establishing the bone reference mark (BRM) includes performing a trial reduction and marking a level of a reamer on an adjacent bone corresponding to which the soft tissue balance is achieved.

4. The method as claimed in claim 1 wherein, the step of performing the secondary scratch fit reaming includes further reaming the bone cavity with a reamer to increase the diameter of the bone cavity.

5. The method as claimed in claim 1 wherein, the step of performing the secondary scratch fit reaming includes performing trial reduction using a final trial for achieving scratch fit.

6. The method as claimed in claim 1 wherein, the method includes selecting a definitive implant having dimensions corresponding to the final trial.

7. The method as claimed in claim 1 wherein, the method includes extracting an old implant using an implant extractor prior to the primary provisional reaming in case the old implant is present in the bone cavity.

8. The method as claimed in claim 1 wherein, the method includes a step of primary provisional trialling post the primary provisional reaming, the primary provisional trialling includes inserting one or more trials one after the other till a perfect fit trial for the bone cavity is found.

9. An implant system for accurate placement of an implant, comprising:
one or more trials;
a reamer comprising a plurality of aligners provided on its body, wherein the reamer is configured to perform a primary provisional reaming (PP reaming) in a bone to obtain a cavity having a depth corresponding to which a maximum range of motion of a joint indicating a soft tissue balance, wherein the reamer is configured to perform a secondary scratch fit reaming (SS reaming) in the bone cavity w.r.t to the diameter of the implant till a scratch fit of an implant is achieved; and
wherein the aligners establish a bone reference mark (BRM) corresponding to a length of a stem of a trial of the one or more trials for which maximum range of motion of the joint is achieved, the length of the stem corresponds to the depth of the cavity.
